SEQUENCE LISTING (8 sequences)

SEQ ID NO: 1 - DNA, 1224 bp, synthetic construct
  source 1..1224: mol_type = other DNA
atggagacagacacactcctgctatgggtactgctgctctgggttccaggttccactggtgactatccatatgatgttccagattatgctggggcccagcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactctccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttag

SEQ ID NO: 2 - DNA, 1224 bp, synthetic construct; identical to SEQ ID NO: 1
  source 1..1224: mol_type = other DNA

SEQ ID NO: 3 - DNA, 1227 bp, synthetic construct
  source 1..1227: mol_type = other DNA
atggagacagacacactcctgctatgggtactgctgctctgggttccaggttccactggtgactatccatatgatgttccagattatgctggggcccagcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactctccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagcgccgcaggccacgttag

SEQ ID NO: 4 - DNA, 1233 bp, synthetic construct
  source 1..1233: mol_type = other DNA
atggagacagacacactcctgctatgggtactgctgctctgggttccatccactggtaacatcgtgctgtatccatatgatgttccagattatgctggggcccagcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactctccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagcgccgcaggccacgttag

SEQ ID NO: 5 - DNA, 1080 bp, synthetic construct
  source 1..1080: mol_type = other DNA
atggagacagacacactcctgctatgggtactgctgctctgggttccatccactggtaacatcgtgctgtatccatatgatgttccagattatgctggggcccagcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactctccgcggctgcaggtcgacgaacaaaaactcatctcagaagaggatctgtag

SEQ ID NO: 6 - DNA, 1131 bp, synthetic construct
  source 1..1131: mol_type = other DNA
atgggagccggcgccacaggcagagccatggacggccctagactgctgctgttgctgctgctgggtgtttccctgggcggcgccaaggaagccgagcagaagctgatctctgaagaggacctggcccagctgacacctaccttctacgacaactcttgtcctaacgtgtccaatatcgtgcgcgacacaatcgtgaacgagctgcggagcgatcctagaatcgcagcttctatcctgcggctgcacttccacgactgcttcgtgaacggctgcgacgccagcatcctgctcgacaatacaaccagcttccggaccgagaaggacgcctttggcaatgccaatagcgccagaggcttccccgtgatcgacagaatgaaagctgctgtggagagcgcctgccccagaaccgtgtcctgcgccgacctgctgacgatcgccgctcaacagagcgtgaccctggctggaggacctagctggcgggtgcccctgggacggagagatagcctgcaggccttcctggacctggccaacgctaacctgcctgcccctttcttcacactgccacagctgaaggacagctttagaaacgtgggcctgaacagatctagcgatctggtcgccctgtccggcggacacaccttcggcaagaaccagtgtagattcatcatggatagactgtataacttctctaacaccggcctccccgaccccacactgaacaccacctacctgcaaacactgaggggcctgtgtccactgaacggcaacctgagcgccctggtggacttcgatctgagaacacccaccatcttcgacaacaagtactacgtgaacctggaagaacagaaaggcctgatccagagcgatcaggagctgttcagcagccctaacgccaccgacaccatccctctcgtgcggagcttcgccaactccacccagacctttttcaacgcctttgtggaggccatggatcggatgggcaacatcacccctctgaccggcacccagggccagatcagactgaactgcagagtggtcaattctaatagcgataacctgattcctgtgtactgcagcatcctggccgccgtggtggtgggcctggttgcttatatcgcctttaagcggtag

SEQ ID NO: 7 - protein, 21 aa, synthetic construct
METDTLLLWVLLLWVPGSTGD

SEQ ID NO: 8 - protein, 23 aa, synthetic construct
METDTLLLWVLLLWVPSTGNIVL

(19) United States
(12) Patent Application Publication
(10) Pub. No.: US 2026/0152727 A1
(43) Pub. Date: Jun. 4, 2026

(54) Modified Viral Genome Compositions and Methods of Production and Use Thereof

(71) Applicant: Board of Regents for the Oklahoma Agricultural and Mechanical Colleges, Stillwater, OK (US)

(72) Inventors: Antonius G.P. OOMENS, Stillwater, OK (US); Pramila LAMICHHANE, Stillwater, OK (US)

(21) Appl. No.: 18/704,723

(22) PCT Filed: Oct. 26, 2022
(86) PCT No.: PCT/US2022/078703

§ 371 Date: Apr. 25, 2024

Related U.S. Application Data
(60) Provisional application No. 63/271,798, filed on Oct. 26, 2021.

(51) Int. Cl. C12N 7/00; C07K 14/71; C12N 9/08; C12N 15/86; G01N 33/532; G01N 33/569

(52) U.S. Cl. CPC C12N 7/00; C07K 14/71; C12N 9/0065; C12N 15/86; C12Y 111/01007; G01N 33/532; G01N 33/56983; C07K 2319/02; C07K 2319/03; C12N 2760/18521; C12N 2760/18543; C12N 2830/002; G01N 2500/00

(57) ABSTRACT

Nucleotide compositions, recombinant vectors, and recombinant viral genomes are disclosed that include a modified horseradish peroxidase (HRP) gene sequence, as well as recombinant viruses that have the modified HRP gene sequence incorporated into the viral genome thereof. The recombinant viruses are utilized in an HRP-based assay to analyze the neutralization potential of candidate antiviral agents, such as antiviral antibodies.

### CROSS-REFERENCE TO RELATED APPLICATIONS/INCORPORATION BY REFERENCE STATEMENT

This application claims benefit under 35 USC § 119 (e) of U.S. Provisional Application Ser. No. 63/271,798, filed Oct. 26, 2021. The entire contents of the above-referenced patent application(s) are hereby expressly incorporated herein by reference.

### STATEMENT REGARDING FEDERALLY SPONSORED RESEARCH OR DEVELOPMENT

This invention was made with Government support under Grant No. 1R21Al128520-01A1 awarded by the National Institutes of Health. The Government has certain rights in the invention.

### BACKGROUND

For a broad range of assays that measure antibody neutralizing potential or the effect of antiviral drugs on virus replication, marker gene expression is used to quantitate viral activity and/or replication levels. Luciferases and fluorescent proteins are currently the most common marker genes utilized for this purpose.

In vitro virus neutralization assays are an important step in determining the potential in vivo efficacy of vaccines or (synthetic) antiviral antibodies. Most recent micro-neutralization assays are based on luciferase or fluorescent read-outs. Viruses of interest are modified by inserting genes encoding luciferase or fluorescent proteins. Following incubation of these modified viruses with antibodies of interest, virus-antibody complexes are then incubated on cells, and the success of neutralization is determined by the level of luminescence or fluorescence. However, these protocols require lysis of cells in which luciferase-expressing viruses are replicating, mixing lysate and luciferase substrate, and reading light output in a luminometer. Whereas this is a popular method, and luciferase substrates are very sensitive, there are a number of technical and cost factors to consider. For example, luciferase substrates are expensive and unstable and have a very short shelf-life, even when stored at −80° C. In addition, the lysis of cells requires proprietary and relatively expensive lysis buffers that are compatible with the luciferase substrates. Also, read-out of these assays requires luminometers, which are expensive and more cumbersome to operate than a typical ELISA reader, and are often not available in poor countries.

Therefore, there is a need in the art for new and improved compositions, assays, and methods for determining the ability of a given antibody to neutralize virus in vitro, and which provides a read-out method that is simpler, faster, cheaper, and does not require a luminometer. It is to said compositions, assays, and methods that the present disclosure is directed.

### BRIEF DESCRIPTION OF THE DRAWINGS

FIG. 1 graphically depicts HRP plasmid constructs generated in accordance with certain non-limiting embodiments of the present disclosure. The construct labeled “pCAG-HRP-TM” represents a commercially available horseradish peroxidase (HRP) plasmid obtained from Addgene (Watertown, MA) and has been assigned SEQ ID NO:1. The construct labeled “pc-HRP-TM (X410)” represents an open reading frame from pCAG-HRP-TM that was subsequently cloned into the inventor's lab vector; this construct has been assigned SEQ ID NO:2. The construct labeled “pc-HRP-TM-no-dilysine (X451)” represents a modification of X410 in which a di-lysine motif at the very carboxy-terminus thereof has been replaced with three arginines (RRR) to avoid potential ER retrieval; this construct has been assigned SEQ ID NO:3. The construct labeled “pc-HRP-TM-no-dilysine-SPO (X455)” represents a construct in which the signal peptide sequence was changed (by mutagenesis) to match the signal peptide of human kappa light chain; this construct has been assigned SEQ ID NO:4. In addition, the upper amino acid sequence indicating the native signal peptide sequence has been assigned SEQ ID NO: 7, while the lower amino acid sequence indicating the mutated signal peptide sequence has been assigned SEQ ID NO:8. The construct labeled “pc-HRP-SEC-SPO (X456)” represents a construct in which the transmembrane domain of HRP was removed to generate a secreted HRP protein; this construct has been assigned SEQ ID NO:5. The final construct labeled “pUC-HRP (X519)” represents a novel construct containing a HRP gene that has been codon optimized for optimal mammalian expression and that has been cloned into the nerve growth factor receptor (NGFR) ORF so that the signal peptide and TMD derived from NGFR are present; this construct was subsequently cloned into the pUC57 vector (gene synthesis by GeneScript, Piscataway, NJ). This last construct has been assigned SEQ ID NO: 6.

FIG. 2 graphically depicts testing to determine if transiently expressed HRP can show ELISA-measurable activity in HEp-2 cells. As described with reference to FIG. 1, the commercially available HRP plasmid (pCAG-HRP-TM; SEQ ID NO:1) was acquired from Addgene (Watertown, MA), and the HRP ORF was also cloned in the pc-DNA based lab vector and named pc-HRP-TM or X410 (SEQ ID NO:2). pCAG-HRP-TM and pc-HRP-TM were transfected into HEp-2 cells. At 26 hpi (hours post infection), cell medium was replaced with OPD ELISA substrate and incubated for 6.5 minutes at room temperature, after which stop solution was added, and OD₄₉₀ was determined. In parallel samples, 0.1% triton-X-100 was added to the substrate. In additional samples, cells were fixed with 2% paraformaldehyde for 5 minutes prior to adding substrate. The results show that transiently expressed HRP was sufficiently active to generate a detectable signal in a standard ELISA assay using a basic OPD substrate. Addition of triton-X-100 did not change signal strength. Mild fixation of transfected cells lowered the signal by an estimated 40-50%.

FIG. 3 graphically depicts HRP-expressing viruses generated in accordance with certain non-limiting embodiments of the present disclosure. The designations associated with the HRP constructs are explained above with reference to FIG. 1.

FIG. 4 graphically depicts a test of signal strength of RSV-HRP (Clone X413) with and without addition of stop solution following substrate incubation. RSV-HRP (X413) was diluted 10-fold, and various dilutions were incubated on HEp-2 cells. At 26 hpi, cell medium was replaced with OPD ELISA substrate and incubated for 6.5 minutes at room temperature, after which stop solution was added and OD₄₉₀ was determined. In parallel samples, stop solution was omitted. The results show that: (1) high levels of HRP activity can be measured after infection of cells with HRP-expressing virus, and (2) HRP activity expressed from a virus can be measured with or without adding stop solution.

FIG. 5 graphically depicts neutralization of RSV-HRP (X459) by anti-preF antibodies (Abs) using standard ELISA reagents. RSV-HRP was incubated with known neutralizing anti-prefusion F Abs D25 (site 0) (REF) and 14402 (site V), or with a control Ab (commercially purchased human IgG). For each antibody (1 mg/ml), a 3-fold dilution series was made starting with a dilution of 1:900 (˜ 1110 ng/ml). Diluted antibodies were incubated with 300 PFU of RSV-HRP for 1.5 hours at 37° C. Next, the virus suspensions were added to HEp-2 cells seeded in 96 well plates, and incubated for 1.5 hours at 37° C. in a CO₂ incubator. After 1.5 hours, the virus inoculum was removed and replaced with regular growth medium, and plates returned to the CO₂ incubator. At 26 hpi, the supernatants were removed and directly (without wash step) replaced with OPD substrate. Substrate was incubated on cells for approximately 5 minutes at room temperature, after which stop solution was added. The plates were then read (OD₄₉₀) in a standard ELISA reader (VersaMax microplate reader, Molecular Devices LLC, San Jose, CA). The data show that HRP expressed from a viral genome could be readily detected and yielded high signals, and was successful in demonstrating in vitro neutralization of RSV by known neutralizing anti-RSV antibodies. This was accomplished rapidly (cell supernatant was directly replaced by substrate without any wash steps), and the signal was read in a standard ELISA reader with standard inexpensive substrate. The OPD substrate made from dry powder yielded identical results as TMB (a liquid substrate).

FIG. 6 graphically depicts neutralization of RSV-HRP-AG (X416) by anti-preF specific antibody D25. The RSV fusion (F) protein is the number one target for vaccine development. To develop a method that can detect exclusively F protein-specific antibodies, an RSV was generated expressing HRP in place of SH, and lacking the G ORF (which was replaced with GFP; see FIG. 3). RSV-HRP-AG thus carries the F protein as its only surface glycoprotein. A neutralization assay was performed identically to the assay used in FIG. 5, but comparing viruses with and without G (X413 and X416, respectively). The data show that a known anti-preF specific antibody (D25) was able to fully neutralize RSV-HRP-AG, whereas a random IgG (purchased commercially) did not. This demonstrates that RSV-HRP-AG can be used to screen or determine levels of RSV F-specific antibodies.

FIG. 7 graphically depicts RSV expressing secreted luciferase ‘Lucia’ (Lucia gene derived from pVITRO2-HYG-Lucia plasmid (InvivoGen, San Diego, CA)). RSV-Lucia was tested in a 96 well format by infecting HEp-2 cells with 300 or 500 PFU/well. At 26 hpi, 25 μl of the cell supernatant was transferred to a new plate, and a series of 2-fold dilutions was made to determine the optimal dilution for luciferase detection. Diluted supernatants were mixed 1:1 with coelenterazine substrate (prepared in acidified methanol and diluted in PBS per manufacturer's instructions), and the plate was read in a luminometer. Dilution of the supernatants from both 300 and 500 PFU amounts yielded an almost linear response, with high signals over background, indicating that a range of supernatant dilutions are appropriate to determine the HRP signal. The signal from undiluted supernatant was too high and exceeded the capacity of the luminometer (not shown).

FIG. 8 graphically demonstrates neutralization of RSV-Lucia by anti-preF Abs D25 and 14402 using coelenterazine substrate. 300 PFU of RSV-Lucia were incubated with D25 and 14402 for 1.5 hours at 37° C. Next, the virus suspensions were added to HEp-2 cells seeded in 96 well plates, and incubated for 1.5 hours at 37° C. in a CO₂ incubator. After 1.5 hours, the virus inoculum was removed and replaced with regular growth medium, and plates returned to the CO₂ incubator. As Lucia luciferase is secreted, at 26 hpi, 25 μl of cell supernatant was transferred to a new plate, and diluted 1:5 in PBS. Plates were then read in a luminometer. Similar to the results of RSV-HRP, complete neutralization was achieved at an Ab dilution of approximately 1:2700, and antibody D25 slightly outperformed 14402.

### DETAILED DESCRIPTION

Certain non-limiting embodiments of the present disclosure are directed to modified viral compositions and kits containing same, as well as methods of producing and using said compositions and kits for determining the antiviral potential of antibodies and/or drugs.

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, l-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively, and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, such as (but not limited to) more than about 85%, 90%, 95%, and 99%. In particular (but non-limiting) embodiments, the object species is purified to essential homogeneity (contaminant species cannot be detected in the composition by conventional detection methods) wherein the composition consists essentially of a single macromolecular species.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

Turning now to the inventive concepts, provided herein are HRP-based constructs for use in vectors, viral genomes, viruses, kits, and methods. Viruses that have been modified to include an HRP-based gene construct in the viral genome thereof can be utilized in rapid and inexpensive in vitro assays for screening antibodies and other candidate agents (such as, but not limited to, small molecule drugs) for neutralization activity as well as other types of antiviral activity. The HRP-based constructs disclosed herein can be included in any virus species that can be engineered to contain foreign sequences and are therefore applicable to many viruses that cause disease in animals and humans.

In an effort to reduce cost and increase simplicity, an in vitro neutralization assay to quantitate viral replication levels has been designed and developed that utilizes this virally-expressed horseradish peroxidase (HRP). Whereas HRP-conjugated secondary antibodies are used worldwide for detection applications such as western blots and ELISAs, the present disclosure is the first application of HRP for antiviral assay purposes. Viruses have been engineered in the present disclosure to express HRP from the viral genome, and in vitro antiviral assays that can be utilized to determine the antiviral activity of candidate agents in a low-cost format (such as, but not limited to, neutralization assays that can determine neutralizing capacity of antiviral agents) have been developed using these engineered viruses. The assays of the present disclosure do not rely on luminescence or fluorescence, utilize inexpensive substrates, and can be read on a basic ELISA plate reader; therefore, these assays constitute a ready-to-go low-cost assay for antiviral agents. Reducing the costs of reagents and the costs associated with purchase and maintenance of specialized equipment will place this antiviral assay method within economic reach of more institutions, including remote locations. In addition, the assay method is readily applicable to any virus for which the genome thereof can be genetically engineered to include the HRP gene constructs disclosed herein.

Certain non-limiting embodiments of the present disclosure are directed to a composition that includes a recombinant horseradish peroxidase (HRP) gene construct. The gene construct includes a nucleotide sequence that encodes at least a portion of a horseradish peroxidase protein. Such nucleotide sequence may be a native HRP sequence or may be modified in one or more ways, so long as the HRP encoded thereby retains HRP activity; for example (but not by way of limitation), the nucleotide sequence encoding the HRP (or portion thereof) may be codon optimized to enhance mammalian expression thereof, may be subjected to site-directed mutagenesis, or may be truncated (such as, but not limited to, to encode a secreted HRP). Alternatively, and/or in addition thereto, the gene construct may include one or more additional encoding sequences that attach one or more heterologous peptides or tags to the HRP. For example (but not by way of limitation), the gene construct may include one or more nucleotide sequences encoding a signal peptide, an HA tag, a transmembrane domain, a myc tag, and the like, so that when the gene construct is expressed, the HRP has the signal peptide, HA tag, transmembrane domain, and/or myc tag attached thereto.

In certain particular (but non-limiting) embodiments, the modified HRP gene construct includes one or more of: a nucleotide sequence flanking an amino terminal end of the HRP gene and encoding a heterologous signal peptide; a nucleotide sequence flanking a carboxyl terminal end of the HRP gene and encoding a heterologous transmembrane domain; and/or a nucleotide sequence encoding a myc epitope tag.

In a particular (but non-limiting) embodiment, the modified HRP gene construct includes a nucleotide sequence flanking an amino terminal end of the HRP gene and encoding at least a portion of a signal peptide of nerve growth factor receptor (NGFR), and a nucleotide sequence flanking a carboxyl terminal end of the HRP gene and encoding at least a portion of a transmembrane domain of NGFR.

In certain particular (but non-limiting) embodiments, the composition comprises a gene construct comprising a sequence represented by one of SEQ ID NOS: 3-6. In a particular (but non-limiting) embodiment, the composition comprises a gene construct comprising a sequence represented by SEQ ID NO:6.

Certain non-limiting embodiments of the present disclosure include a recombinant vector comprising any of the recombinant horseradish peroxidase (HRP) gene construct-containing compositions disclosed or otherwise contemplated herein. A tremendous number of plasmid vectors are widely known and commercially available in the art that can be utilized as the backbone into which the HRP gene constructs of the present disclosure are inserted. The only requirement for said plasmid vector is that there be restriction sites present that allow for removal of the HRP gene construct when modifying a viral genome to incorporate the HRP gene construct therein. Non-limiting examples of plasmid/vector backbones into which the HRP gene construct has been cloned (as described in the Example) are pCAG, pUC57, and a pc-DNA based lab vector. However, numerous other examples of vectors that can be utilized as described herein are widely known and commercially available; therefore, no further description thereof is deemed necessary.

In certain particular (but non-limiting) embodiments, the modified HRP gene construct includes one or more of: a nucleotide sequence flanking an amino terminal end of the HRP gene and encoding a heterologous signal peptide; a nucleotide sequence flanking a carboxyl terminal end of the HRP gene and encoding a heterologous transmembrane domain; and/or a nucleotide sequence encoding a myc epitope tag.

In a particular (but non-limiting) embodiment, the modified HRP gene construct present in the vector includes a nucleotide sequence flanking an amino terminal end of the HRP gene and encoding at least a portion of a signal peptide of nerve growth factor receptor (NGFR), and a nucleotide sequence flanking a carboxyl terminal end of the HRP gene and encoding at least a portion of a transmembrane domain of NGFR.

In certain particular (but non-limiting) embodiments, the modified HRP gene construct present in the recombinant vector comprises a sequence represented by one of SEQ ID NOS: 3-6. In a particular (but non-limiting) embodiment, the modified HRP gene construct present in the recombinant vector comprises a sequence represented by SEQ ID NO:6.

Certain non-limiting embodiments of the present disclosure are directed to a recombinant viral genome comprising an open reading frame that includes any of the recombinant horseradish peroxidase (HRP) gene constructs disclosed or otherwise contemplated herein. As stated herein above, the gene construct includes a native or modified nucleotide sequence that encodes at least a portion of a horseradish peroxidase protein, including modified nucleotide sequences that have been codon optimized, subjected to site-directed mutagenesis, truncated, and/or conjugated to one or more additional nucleotide sequences (such as, but not limited to, nucleotide sequence(s) encoding a signal peptide, an HA tag, a transmembrane domain, and/or myc tag, and the like).

In certain particular (but non-limiting) embodiments, the open reading frame incorporated in the recombinant viral genome includes a nucleotide sequence encoding at least a portion of a horseradish peroxidase (HRP) that has any of the native or modified sequences disclosed or otherwise contemplated herein (such as, but not limited to, a sequence that has been codon optimized for mammalian expression), in combination with one or more of: a nucleotide sequence flanking an amino terminal end of the HRP gene and encoding a heterologous signal peptide; a nucleotide sequence flanking a carboxyl terminal end of the HRP gene and encoding a heterologous transmembrane domain; and/or a nucleotide sequence encoding a myc epitope tag.

In a particular (but non-limiting) embodiment, the open reading frame includes a nucleotide sequence flanking an amino terminal end of the HRP gene and encoding at least a portion of a signal peptide of nerve growth factor receptor (NGFR), and a nucleotide sequence flanking a carboxyl terminal end of the HRP gene and encoding at least a portion of a transmembrane domain of NGFR.

In certain particular (but non-limiting) embodiments, at least a portion of the open reading frame present in the recombinant viral genome is represented by one of SEQ ID NOS: 1-6. In certain particular (but non-limiting) embodiments, at least a portion of the open reading frame present in the recombinant viral genome is represented by one of SEQ ID NOS: 3-6. In a particular (but non-limiting) embodiment, the open reading frame present in the viral genome comprises a sequence represented by SEQ ID NO:6.

Certain non-limiting embodiments of the present disclosure are directed to a recombinant virus that includes any of the recombinant viral genomes disclosed or otherwise contemplated herein. As stated herein above, the recombinant viral genome has a HRP gene construct incorporated therein, wherein the gene construct includes a native or modified nucleotide sequence that encodes at least a portion of a horseradish peroxidase protein, including modified nucleotide sequences that have been codon optimized, subjected to site-directed mutagenesis, truncated, and/or conjugated to one or more additional nucleotide sequences (such as, but not limited to, nucleotide sequence(s) encoding a signal peptide, an HA tag, a transmembrane domain, and/or myc tag, and the like).

In certain particular (but non-limiting) embodiments, the recombinant virus includes a modified horseradish peroxidase (HRP) gene construct incorporated in a genome of the virus, wherein the modified HRP gene construct comprises an open reading frame comprising a nucleotide sequence encoding at least a portion of a horseradish peroxidase (HRP) (including any of the native or modified HRP-encoding sequences disclosed or otherwise contemplated herein, such as a codon optimized sequence), and at least one of a nucleotide sequence flanking an amino terminal end of the HRP gene and encoding a heterologous signal peptide and/or a nucleotide sequence flanking a carboxyl terminal end of the HRP gene and encoding a heterologous transmembrane domain.

In a particular (but non-limiting) embodiment, the open reading frame present in the viral genome of the recombinant virus includes a nucleotide sequence flanking an amino terminal end of the HRP gene and encoding at least a portion of a signal peptide of nerve growth factor receptor (NGFR), and a nucleotide sequence flanking a carboxyl terminal end of the HRP gene and encoding at least a portion of a transmembrane domain of NGFR.

In certain particular (but non-limiting) embodiments, at least a portion of the open reading frame present in the viral genome of the recombinant virus is represented by one of SEQ ID NOS: 1-6. In certain particular (but non-limiting) embodiments, at least a portion of the open reading frame present in the viral genome of the recombinant virus is represented by one of SEQ ID NOS: 3-6. In a particular (but non-limiting) embodiment, the open reading frame present in the viral genome comprises a sequence represented by SEQ ID NO:6.

The recombinant virus (or viral genome thereof) may be any virus that is capable of being genetically modified, and for which an antiviral agent (such as, but not limited to, a neutralizing antibody or small molecule agent) having antiviral activity against the virus is desired. Non-limiting examples of viruses that may be genetically modified in accordance with the present disclosure include adenoviruses, astroviruses, coronaviruses, Coxsackie viruses, cytomegaloviruses (CMV), echoviruses, encephalitis viruses, enteroviruses, Epstein-Barr viruses (EBV), erythroviruses, hantaviruses, hepatitis viruses, herpes viruses, human immunodeficiency viruses (HIV), influenza viruses, noroviruses, papilloma viruses, parainfluenza viruses, paramyxoviruses, polio viruses, rabies viruses, respiratory syncytial viruses (RSV), rhinoviruses, rotaviruses, rubella viruses, rubeola viruses, Varicella-Zoster viruses, West Nile viruses, and Zika viruses, and the like. Particular non-limiting examples of viruses that may be utilized in accordance with the present disclosure include respiratory syncytial virus (RSV), severe acute respiratory syndrome coronavirus 2 (SARS-COV-2), influenza virus, HIV, Hepatitis B virus, Hepatitis C virus, Epstein-Barr virus, Herpes Virus (HSV), CMV, Rubella virus, and the like. [Any examples you want to add or remove from these two lists?]

In a particular (but non-limiting) embodiment, the recombinant virus is RSV. The genome of the RSV may retain all native sequences, or the viral genome may be engineered to modify the coding sequence of one or more proteins thereof and/or modified to lack an entire coding sequence for one or more proteins thereof. For example (but not by way of limitation), the RSV genome may lack a gene encoding an SH protein and/or a gene encoding a G protein (or the gene(s) may be otherwise modified to prevent production of the SH and/or G protein). Utilizing an RSV that lacks G protein allows for analysis of candidate agents that specifically target F protein.

Certain non-limiting embodiments of the present disclosure are directed to a method that comprises incorporating any of the modified horseradish peroxidase (HRP) gene constructs disclosed or otherwise contemplated herein into a viral genome of any of the viruses disclosed or otherwise contemplated herein to produce a modified viral genome. Methods of inserting a nucleotide sequence into a viral genome are well known in the art, and kits for performing said insertion are widely available commercially. Therefore, no further discussion of any steps involved in said method are deemed necessary.

Certain non-limiting embodiments of the present disclosure are directed to a kit that includes one or more of the compositions disclosed or otherwise contemplated herein. For example (but not by way of limitation), the kit may include one or more of any of the modified HRP gene constructs, one or more of any of the recombinant vectors, one or more of any of the recombinant viral genomes, and/or one or more of any of the recombinant viruses disclosed or otherwise contemplated herein.

The kits of the present disclosure may be provided with one or more additional reagents that can be used in any of the methods of the present disclosure. For example, but not by way of limitation, the kits may include plasmid/vector maps (indicating, for example (but not by way of limitation), positioning of restriction site(s) with respect to the HRP gene construct), viral cDNA, at least one microplates, at least one medium, at least one detectable substrate for HRP, a stop solution, and the like, as well as any combinations thereof. ELISA substrates that can be utilized as detectable substrates for HRP are well known in the art and commercially available; non-limiting examples thereof include o-phenylenediamine dihydrochloride (OPD); 3,3′,5,5′-tetramethylbenzidine (TMB); 2,2′-azino-bis(3-ethylbenzothiazoline-6-sulfonic acid) (ABTS); and the like, as well as any combinations thereof. [Do you have any other examples of substrates that should be added?]

Also, the components/reagents present in the kits may each be in separate containers/compartments, or various components/reagents can be combined in one or more containers/compartments, depending on the sterility, cross-reactivity, and stability of the components/reagents. In addition, the kit may be disposed in any packaging that allows the components present therein to function in accordance with the present disclosure. In certain non-limiting embodiments, the kit further comprises a sealed packaging in which the components are disposed.

In addition, the kit can further include a set of written instructions explaining how to use one or more components of the kit. A kit of this nature can be used in any of the methods described or otherwise contemplated herein.

Certain non-limiting embodiments of the present disclosure are directed to a method in which any of the recombinant viruses described or otherwise contemplated herein are combined substantially simultaneously or wholly or partially sequentially with at least one candidate agent and at least one cell to form a mixture. The mixture is then cultured under conditions that allow for infection of the at least one cell by the recombinant virus and expression by the at least one cell of the modified HRP from a genome of the recombinant virus. The at least one cell is then contacted with any of the substrates for HRP disclosed or otherwise contemplated herein, and an optical density (OD) is measured to detect signal generated by the HRP substrate and calculate an HRP expression level. Then it is determined that the at least one candidate agent is a potential antiviral agent if the HRP expression level observed is lower than an HRP expression level obtained in the absence of the at least one candidate agent.

Any cell type capable of being cultured in accordance with the methods disclosed herein and capable of being infected by the virus and producing the HRP encoded by the viral genome can be utilized in accordance with the present disclosure. Non-limiting examples of cell types that may be utilized in accordance with the present disclosure include HEp-2 cells, ______. [what other examples of cell types can we provide?]

The three compositions (i.e., recombinant virus, candidate agent, and cell) can be contacted simultaneously or wholly or partially sequentially. When contacted in a sequential manner, the recombinant virus may be contacted with the candidate agent first, and then the suspension containing the recombinant virus and candidate agent may be contacted with the cell; this order of addition allows for the determination of whether or not the candidate agent is effective at preventing the virus from infecting the cell. Alternatively, the recombinant virus may be contacted with the cell first (so that the cell becomes infected with the virus prior to addition of the candidate agent), and then the candidate agent added to analyze other antiviral activities that the candidate agent may possess. In this manner, the ability of an antiviral agent to both prevent/reduce the occurrence of infection as well as treat an existing infection can be analyzed.

Certain particular (but non-limiting) embodiments of the present disclosure are directed to a method comprising the steps of: (i) contacting any of the recombinant viruses disclosed or otherwise contemplated herein with at least one candidate agent (such as, but not limited to, an antibody or small molecule agent) and incubating under conditions that allow the at least one candidate agent to interact with the recombinant virus; (ii) contacting the recombinant virus with at least one of any of the cells disclosed or otherwise contemplated herein to form a mixture; (iii) culturing the mixture under conditions that allow for infection of the at least one cell by the recombinant virus and expression by the at least one cell of the modified HRP from a genome of the recombinant virus; (iv) contacting the at least one cell with any of the substrates for HRP disclosed or otherwise contemplated herein; (v) measuring an optical density (OD) to detect signal generated by the HRP substrate and calculating an HRP expression level based on the OD; and (vi) determining that the at least one candidate agent is a potential antiviral agent if the HRP expression level observed in step (v) is lower than an HRP expression level obtained in the absence of the at least one candidate agent.

Certain non-limiting embodiments of the present disclosure are directed to a method that comprises the steps of: (i) contacting any of the recombinant viruses disclosed or otherwise contemplated herein with at least one candidate agent (such as, but not limited to, an antibody or small molecule agent) to form a suspension and incubating the suspension under conditions that allow the at least one candidate agent to interact with the recombinant virus; (ii) contacting and culturing at least one of any of the cells disclosed or otherwise contemplated herein attached to a surface with the suspension under conditions that allow for infection of the at least one cell by the recombinant virus; (iii) substantially removing the suspension and culturing the at least one cell in the presence of growth medium under conditions that allow for expression of the modified HRP from a genome of the recombinant virus; (iv) substantially removing a supernatant containing the growth medium and contacting the at least one cell with any of the substrates for HRP disclosed or otherwise contemplated herein; (v) measuring an optical density (OD) to detect signal generated by the HRP substrate and calculating an HRP expression level based on the OD; and (vi) determining that the at least one candidate agent is a potential antiviral agent if the HRP expression level observed in step (v) is lower than an HRP expression level obtained in the absence of the at least one candidate agent.

In certain particular (but non-limiting) embodiments, the method further comprises the step of adding a stop solution between steps (iv) and (v).

Certain non-limiting embodiments of the present disclosure are directed to a method that comprises the steps of: (i) contacting any of the cells disclosed or otherwise contemplated herein with any of the recombinant viruses disclosed or otherwise contemplated herein and at least one candidate agent (such as, but not limited to, an antibody or small molecule agent or drug) to form a mixture; (ii) culturing the mixture under conditions that allow for infection of the at least one cell by the recombinant virus and expression by the at least one cell of the modified HRP from a genome of the recombinant virus; (iii) contacting the at least one cell with any of the substrates for HRP disclosed or otherwise contemplated herein; (iv) measuring an optical density (OD) to detect signal generated by the HRP substrate and calculating an HRP expression level based on the OD; and (v) determining that the at least one candidate agent is a potential antiviral agent if the HRP expression level observed in step (v) is lower than an HRP expression level obtained in the absence of the at least one candidate agent.

In certain particular (but non-limiting) embodiments, the at least one cell is contacted with the recombinant virus prior to the addition of the at least one candidate agent.

In certain particular (but non-limiting) embodiments, the method may further comprise the step of adding a stop solution between steps (iii) and (iv).

In addition to each and every embodiment disclosed herein, it will be understood that virus-induced HRP expression can also be achieved by expressing HRP from a cell line in a fashion whereby the virus provides a trigger for HRP expression. In one non-limiting example, a cell line is generated expressing HRP from a Tet-Responsive-Element (TRE-HRP). The virus then expresses the Tet transactivator protein from the viral genome, which triggers HRP expression in cells infected by virus. Other mechanisms such as the cre-lox system may also be employed to achieve virus-triggered HRP expression from a cell line.

### Example

An Example is provided hereinbelow. However, the present disclosure is to be understood to not be limited in its application to the specific experimentation, results, and laboratory procedures disclosed herein after. Rather, the Example is simply provided as one of various embodiments and are meant to be exemplary, not exhaustive.

It is well known that sufficient in vivo levels of an antiviral antibody are needed for protection against a virus; however, the ability of a given antibody to neutralize virus in vitro is a better predictor of antibody performance in vivo than in vivo antibody levels alone. Different techniques have been employed to determine antibody neutralizing potential, including the traditional plaque assay and TCID₅₀; however, both of these techniques are labor-intensive. Most recent techniques are based on the ELISA platform, with the vast majority utilizing luciferase activity or fluorescence levels as a read-out. Recombinant viruses carrying the luciferase gene (especially Firefly and Renilla luciferases) or fluorescent marker genes have been constructed for a variety of disease-causing viruses. The luciferase protocol requires lysis of cells in which luciferase-expressing viruses are replicating, mixing lysate and luciferase substrate, and reading light output in a luminometer. Whereas this is a popular method, and luciferase substrates are very sensitive, there are a number of technical and cost factors to consider. For example, luciferase substrates are expensive and unstable and have a very short shelf-life, even when stored at −80° C. In addition, the lysis of cells requires proprietary and relatively expensive lysis buffers that are compatible with the luciferase substrates. Also, read-out of these assays requires luminometers, which are expensive and more cumbersome to operate than a typical ELISA reader, and are often not available in poor countries.

To design a read-out method which is simpler, faster, cheaper, and does not require a luminometer, the gene encoding horseradish peroxidase (HRP) was cloned into the RSV genome, and its ability to demonstrate the neutralization potential of known neutralizing anti-RSV antibodies (Abs) was tested. In addition, for reasons of comparison, an RSV expressing a novel secreted luciferase gene was generated. The results provided herein below demonstrate that the HRP-based assay constitutes an inexpensive and attractive method to measure neutralization potential in vitro that is applicable to many viruses.

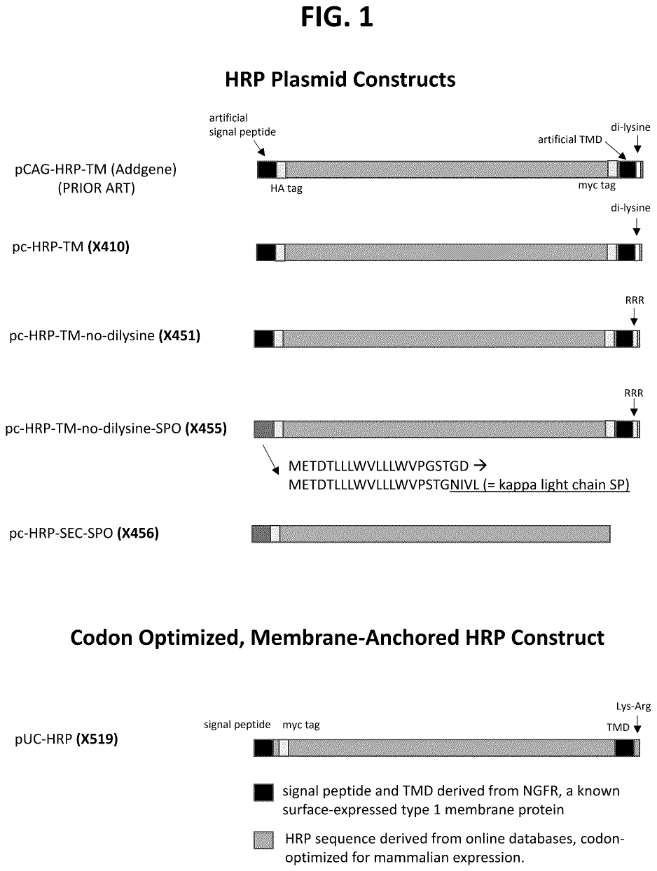

Acquisition and design of HRP genes. A plasmid containing a modified HRP gene (pCAG-HRP-TM; SEQ ID NO:1) was acquired from Addgene (Watertown, MA) (FIG. 1) for testing of concept. In this plasmid, the HRP-encoding nucleotides are flanked by an artificial signal peptide and an HA tag on the amino-terminal end, and a transmembrane domain and myc tag on the carboxy-terminal end. The HRP ORF from pCAG-HRP-TM was cloned in a pcDNA-based expression plasmid and named pc-HRP-TM (X410; SEQ ID NO:2).

The HRP-TM ORF in plasmid X410 was slightly modified by replacing a di-lysine motif at the very carboxy-terminus with three arginines to avoid potential ER retrieval. The resulting plasmid was designated X451 (SEQ ID NO:3). The di-lysine modified HRP-TM ORF was later found to yield similar HRP activity as X410 (data not shown).

Due to placement of the HA tag in X410, there was a concern that the signal peptide may not function optimally. Therefore, the signal peptide sequence was changed (by mutagenesis) to match the signal peptide of human kappa light chain. The resulting construct was designated X455 (SEQ ID NO:4).

Finally, the transmembrane domain of HRP was removed to generate a secreted HRP protein. The resulting construct was designated X456 (SEQ ID NO:5). In some non-limiting aspects, secreted marker proteins can hold certain advantages over cell-bound marker proteins.

The above constructs were derived from a commercially available HRP to enable expeditious testing of the potential of the novel method. After early testing rounds (see later figures) and positive results were obtained, a novel HRP gene was designed and generated. The HRP sequence was derived from online sources and codon optimized for mammalian expression, and was used to clone into the nerve growth factor receptor (NGFR) ORF from which the ectodomain was deleted. NGFR is a known highly surface-expressed protein that contains its own signal peptide (SP) and transmembrane domain (TMD). Whereas the SP and TMD of NGFR were left intact, its ectodomain was replaced with nucleotides encoding the newly-designed HRP. In addition, a myc epitope tag was inserted a few amino acids downstream of the SP cleavage site. The resulting lab-designed membrane-anchored HRP was designated X519 (SEQ ID NO: 6).

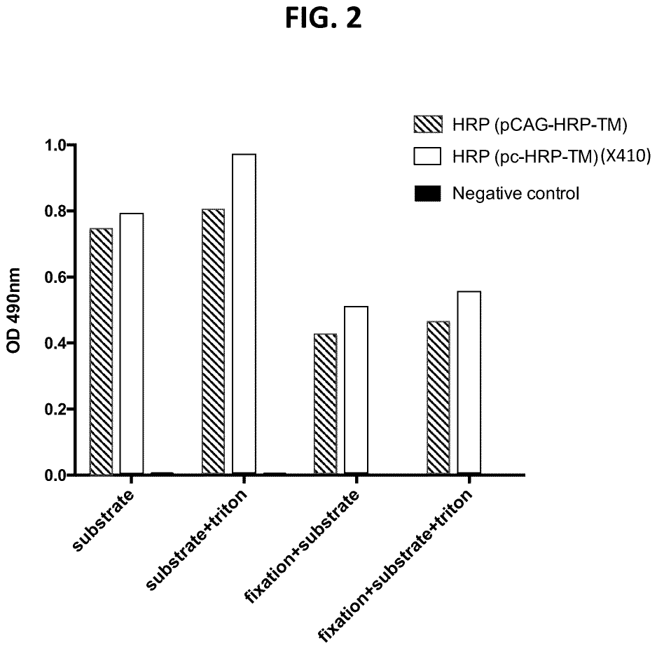

Testing to determine if transiently expressed HRP show ELISA-measurable activity in HEp-2 cells: Plasmids pCAG-HRP-TM and X410 were transfected into HEp-2 cells in 12-well plates to examine whether the expressed HRP could yield a signal using the standard and inexpensive ELISA substrate o-phenylenediamine dihydrochloride (OPD). At 26 hpt (hours post transfection), cell supernatants were directly replaced with OPD substrate (400 μl/well). This was done in the presence or absence of 0.1% triton, to examine if membrane permeabilization would enhance the signal. In parallel wells, cells were fixed with 2% paraformaldehyde prior to incubation with OPD substrate with or without 0.1% triton (FIG. 2). To stop the reaction after a 6.5 minute incubation period, 50 μl was transferred to a 96 well plate containing 50 μl stop solution (3M H₂SO₄) per well. Next, the OD₄₉₀ was determined in a standard ELISA reader. All conditions yielded an ELISA signal well above background (mock-transfected cells). The highest OPD signals were obtained without prior fixation.

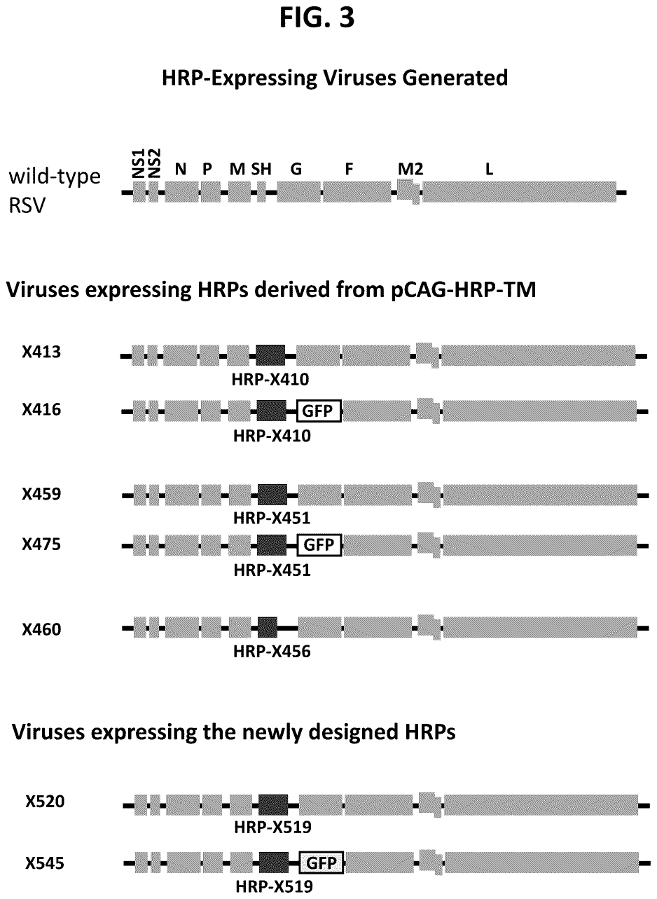

Construction of HRP-containing RSV: Remote-cutting BsmBI restriction sites were created before and after the various HRP ORFs, and the ORFs were then cloned into an RSV cDNA (strain A2) in which the SH ORF was replaced with a BsmBI cloning cassette (FIG. 3). The SH location was used because, in the inventor's experience, artificial genes inserted there are highly expressed. Also, the SH protein is dispensable and, in contrast to G and F, not a major protective antigen. Use of the BsmBI sites ensured that the RSV is fully wild-type, other than an exact replacement of the SH ORF with that of HRP. All of the various HRPs shown in FIG. 1 were cloned into the SH location of RSV cDNA. Using reverse genetics as described previously (Batonick et al. (J Virol (2008) 82:8664-8672); Baviskar et al. (J Virol (2013) 87:10730-10741); Mitra et al. (J Virol (2012) 86:4432-4443)), infectious viruses were recovered from the HRP-containing cDNA, and were collectively designated RSV-HRP. The various RSV-HRP were designated X413, X416, X459, X475, X460, X520, and X545 (see FIG. 3); the figure also indicates which HRP is present in each virus. RSV-HRP titers of 1×10⁷ PFU/ml were readily obtained, although they appeared to amplify at rates slightly lower than wt RSV.

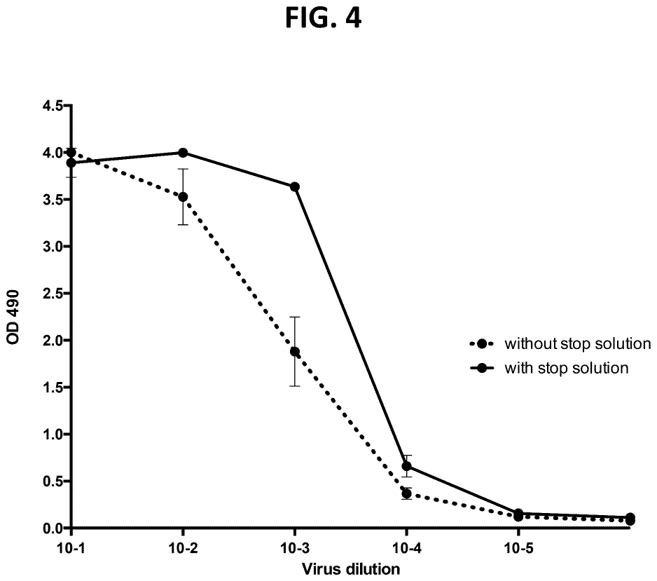

The ability of RSV to express functionally competent HRP from the viral genome was tested in a dose-response experiment (FIG. 4). HEp-2 cells were infected with varying amounts RSV-HRP (X413). At 26 hpi, cell supernatant was replaced with OPD substrate. After 6.5 minute incubation, stop solution was added, and HRP signals (OD₄₉₀) were read in an ELISA reader. In parallel wells, the OD₄₉₀ was determined without added stop solution. Under both conditions, the results showed a dose-dependent response, indicating that the technique was suitable for in vitro virus neutralization.

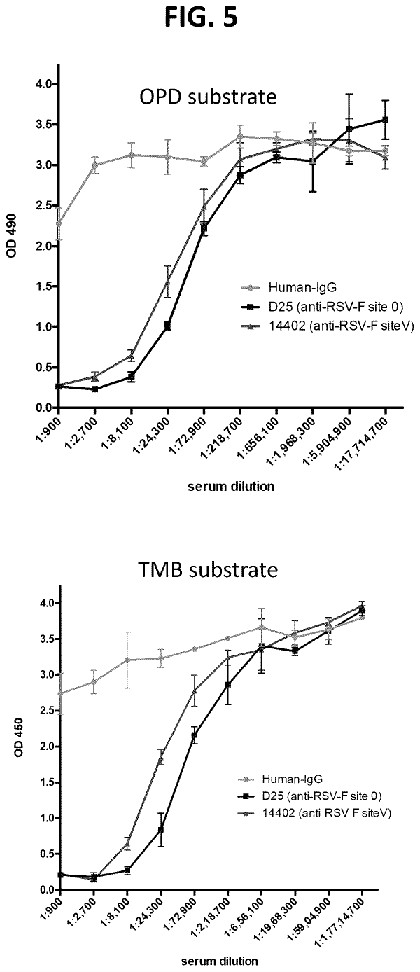

Neutralization of RSV-HRP by anti-preF antibodies (Abs) using standard ELISA reagents: Having generated HRP-expressing RSV, these viruses were tested for their ability to demonstrate RSV neutralization by known anti-RSV Abs. To establish the minimal amount of PFU to demonstrate RSV-HRP-based neutralization, preliminary tests were performed showing that below 200-300 PFU, the signal-to-background ratio becomes too low (not shown). Although higher amounts (up to 1000 PFU) were successful, to maintain sensitivity, 300 PFU were used in the experiments shown here. This number is similar to the number of PFUs used by other groups. RSV-HRP was incubated with known neutralizing anti-preF Abs D25 (site 0) and 14402 (site V), or with a control antibody (commercially purchased human IgG). For each antibody (1 mg/ml), a 3-fold dilution series was made starting with a dilution of 1:900 (˜ 1110 ng/ml). Diluted antibodies were incubated with 300 PFU of RSV-HRP for 1.5 hours at 37° C. Next, the virus suspensions were added to HEp-2 cells seeded in 96 well plates, and incubated for 1.5 hours at 37° C. in a CO₂ incubator. After 1.5 hours, the virus inoculum was removed and replaced with regular growth medium, and plates returned to the CO₂ incubator. At 26 hpi, the supernatants were removed and directly (without wash step) replaced with OPD substrate. Substrate was incubated on cells for approximately 5 minutes at room temperature, after which stop solution was added. The plates were then read (OD₄₉₀) in a standard ELISA reader (VersaMax, Molecular Devices). The data showed that D25 and 14402 were similar in their capacity to neutralize RSV-HRP (FIG. 5, top panel), with D25 slightly outperforming 14402. The latter agrees with Crank et al. (Science (2019) 365:505-509), which shows that site 0 is the most neutralization sensitive site. The highest dilution at which the antibodies negatively impacted RSV-HRP infectivity was approximately 1:200,000 (5 ng/ml). At a concentration of approximately 1:2,700 (370 ng/ml), both antibodies completely neutralized 300 PFU of RSV-HRP. Ab D25 appeared to slightly outperform Ab 14402 in neutralizing capacity. The assay was also performed with the commercially prepared high-sensitivity ELISA substrate 3,3′,5,5′-tetramethylbenzidine (TMB) (Pierce PI34021) (FIG. 5, bottom panel). TMB was similarly applied, but incubated on cells for 30 minutes at room temperature per manufacturer's instructions, before adding stop solution. TMB substrate yielded very similar results and did not show a higher sensitivity than OPD. Together, the data show that the HRP-based method was able to demonstrate the neutralizing potential of two known anti-preF Abs, using two distinct standard ELISA substrates. Whereas the TMB substrate is more expensive than OPD made from powder, OPD was equally effective under these conditions.

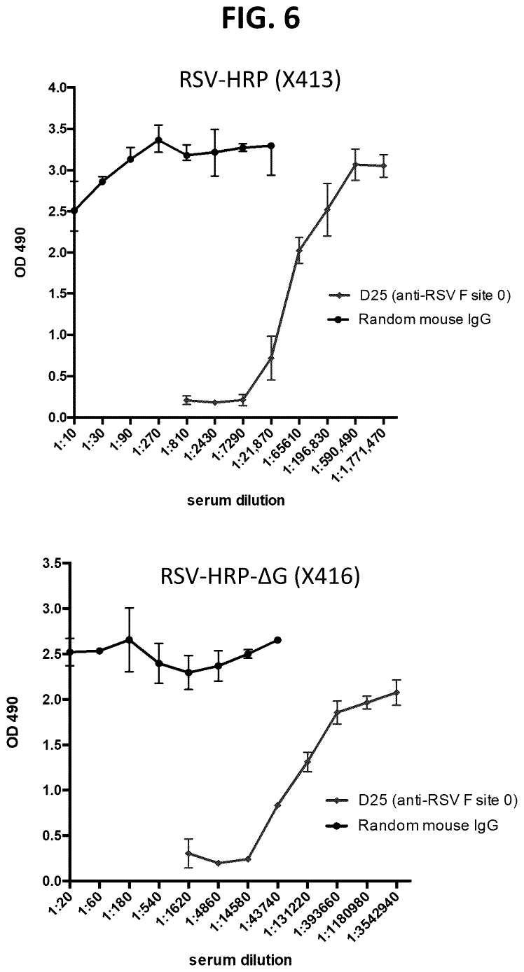

An RSV-HRP variant lacking the G ORF, for screening or determination of antibody (Ab) levels specific for the RSV F protein (FIG. 6): Neutralization of RSV-HRP-AG (X416) by anti-preF specific antibody D25. Due to its essential nature among the glycoproteins, the RSV fusion (F) protein is the number one target for vaccine development. To develop a method that can detect exclusively F protein-specific antibodies, RSV-HRP was modified to lack the G ORF (which was replaced with GFP) and designated X416; see FIG. 3. RSV-HRP-AG thus carries the F protein as its only surface glycoprotein. Two additional versions lacking G were also generated, and designated X475 and X545, see FIG. 3. However, RSV-HRP-AG version X416 was used to demonstrate utility. A neutralization assay was performed identically to the assay used in FIG. 5, but comparing viruses with and without G (X459 and X416, respectively).

The data show that a known anti-preF specific antibody (D25) was able to fully neutralize both RSV-HRP (which contains G) and RSV-HRP-AG (which lacks G), whereas a random IgG (purchased commercially) did not. This demonstrates that RSV-HRP-AG can be used to screen or determine levels of RSV F-specific antibodies.

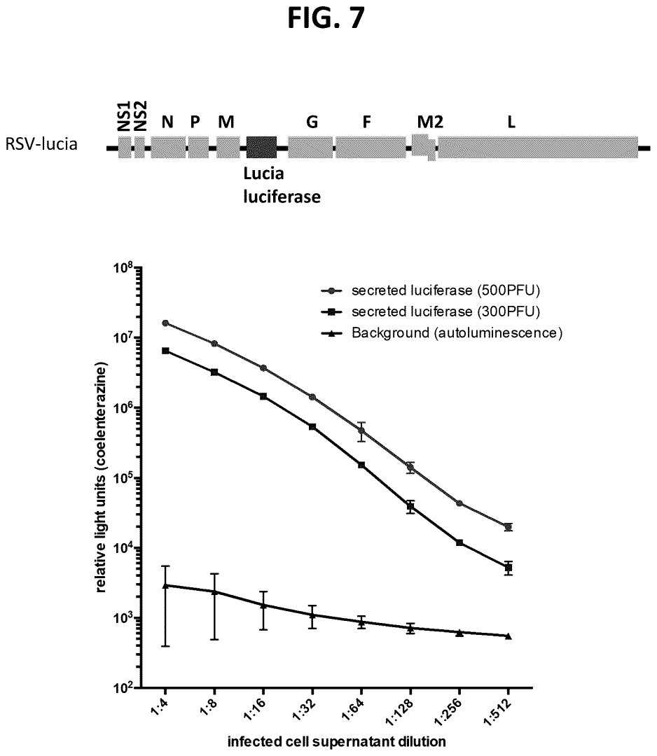

Construction of secreted luciferase-containing RSV (FIG. 7): As a comparison to RSV-HRP, but also as a potential improvement over previous neutralization methods based on standard, non-secreted, luciferases, an RSV expressing a secreted luciferase was generated. Plasmid pVITRO2-HYG-Lucia was acquired from InvivoGen (San Diego, CA). The Lucia luciferase is a naturally secreted luciferase that utilizes coelenterazine-based substrates. Using engineered BsmBI sites, the Lucia ORF was cloned into the SH location of RSV (FIG. 2), and an infectious virus was recovered from cDNA as described above. The resulting virus was named RSV-Lucia. Initially, RSV-Lucia was tested in a 96 well format by infecting HEp-2 cells with 300 or 500 PFU/well (FIG. 4). At 26 hpi, 25 μl of the cell supernatant was transferred to a new plate, and a series of 2-fold dilutions were made to determine the optimal dilution for luciferase detection. Diluted supernatants were mixed 1:1 with coelenterazine substrate (prepared in acidified methanol and diluted in PBS per manufacturer's instructions), and the plate was read in a luminometer. Dilution of the supernatants from both 300 and 500 PFU amounts yielded an almost linear response, with high signals over background, indicating that a range of supernatant dilutions are appropriate to determine the HRP signal. The signal from undiluted supernatant was too high and exceeded the capacity of the luminometer (not shown).

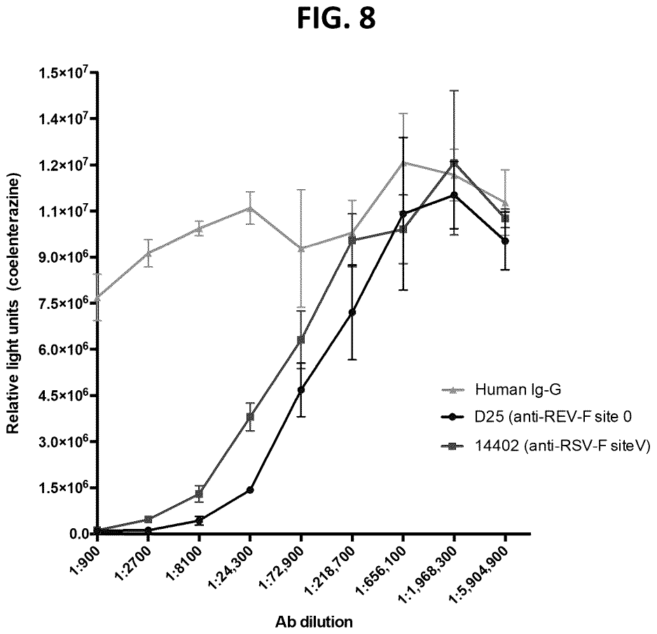

Neutralization of RSV-Lucia by anti-preF Abs D25 and 14402 using coelenterazine substrate: The ability of RSV-Lucia to demonstrate RSV in vitro neutralization by known RSV antibodies was examined next (FIG. 8). An equal amount of PFU (300) of RSV-Lucia were incubated as above with D25 and 14402. As Lucia luciferase is secreted, at 26 hpi, 25 μl of cell supernatant was transferred to a new plate, and diluted 1:5 in PBS. Plates were then read in a luminometer. When graphed, the results were similar to those of the RSV-HRP assay. Similar to the results of RSV-HRP, complete neutralization was achieved at an antibody dilution of approximately 1:2700. However, the highest dilution at which the antibodies negatively impacted RSV infectivity was between 1:200,000 (5 ng/ml) and 1:600,000 (1.7 ng/ml). As a consequence, the antibody dilutions at which half of the virus was neutralized was slightly higher for RSV-Lucia. As seen above, D25 again slightly outperformed 14402 in neutralizing capacity.

### Discussion

HRP is an enzyme well-known for its use in ELISA and enhanced chemi-luminescence (i.e., western blot) assays. As such, relatively cheap HRP substrates are widely commercially available. In an effort to develop a method to gauge neutralizing potential of unknown RSV antibodies (as well as other agents) that is both simple and inexpensive, the HRP gene was incorporated into the SH locus of RSV. Previously characterized neutralizing Abs D25 and 14402, which recognize distinct sites in preF, were then used to test whether HRP, when expressed from within the viral genome, could serve as a marker gene in novel in vitro neutralization assays using a standard ELISA reader. Data generated with HRP expressing viruses were compared to viruses expressing secreted luciferase to demonstrate that HRP expressed from the viral genome is an effective, rapid, and low-cost method to examine antiviral antibodies or drugs.

HRP-based in vitro neutralization assays possess several advantages over previously used methods. Luciferase substrates are very sensitive and have been successfully used for in vitro neutralizations previously. However, the HRP-based assay also relies on direct measurement of the level of a marker gene, and possess advantages related to reduced cost and improved convenience, while still providing an adequate output that allows comparison of neutralizing capacity. In addition, luciferase substrates are expensive and unstable and need to be stored at ultralow temperature; even then, these substrates only have a shelf-life of a few weeks. In contrast, HRP substrates are cheaper and highly stable. TMB can be stored at room temperature and has a very long shelf-life. OPD can be bought as tablets or powder; while this substrate needs to be stored at −20° C., it can last for years at such storage temperature. Even a basic and very inexpensive substrate made from OPD powder generated usable neutralization curves. In addition to its low cost, the HRP-based assay is fast and simple relative to luciferase assays. For the latter, cells need to be lysed in often proprietary and relatively expensive buffers, centrifuged to remove debris, and then diluted and transferred to a luminescence read-out plate, which are specialty plates and also expensive. For the HRP assay, regular 96-well plates suffice, and only two steps, and no additional plates, are required for read-out. The latter consists only of replacing the cell supernatant with substrate solution (no wash steps necessary) and adding stop solution. The last significant advantage of the HRP assay is that it can be read in any standard ELISA reader, whereas luciferase-based assays require a luminometer. Luminometers are more expensive to acquire and more cumbersome to operate than a standard ELISA plate reader; in addition, luminometers are not as available as ELISA plate readers, especially in underprivileged settings or countries where RSV can be a major burden.

Secreted luciferase-based in vitro neutralization: The secreted luciferase assay is, for reasons stated above, more expensive and more complex to operate than the HRP-based assay. Its advantages over previous luciferase-based assays are that the protocol is a little faster and cheaper (cells do not need to be lysed), and that the reaction can be read multiple times as the luciferase is harvested from the supernatant. In this case, a small amount of cell supernatant at 26 hpi was already saturating to the luminometer and required dilution prior to reading. Relative to the HRP-based assay, the sensitivity of the secreted luciferase can be exploited by reading earlier and shortening incubation time of the assay.

Comparison of neutralization methods. The neutralizing potentials of D25 and 14402 were slightly lower in the secreted-luciferase assay than in the HRP-based assay. One potential reason is that non-infectious to infectious particle ratios can vary between batches of virus, and may be higher for RSV-HRP than for RSV-Lucia, leading to more antibodies being adsorbed by non-infectious particles in the case of RSV-HRP. This ratio and other variables (such as origin, quality, and purification status of the virus; cell types used to grow the virus; amount of input PFU; quality and purity of the Ab; and type and passage number of the cells used in the assay) also make it difficult to compare absolute neutralization titers between labs. In that regard, there are efforts within the RSV field to standardize all components such that the relative efficiency of novel antibodies can be more readily assessed. Regardless of whether such standardization can be achieved globally, the HRP-based assay constitutes a rapid and cheap assay to compare the neutralizing capacity of a novel antibody to one previously characterized.

RSV-HRP propagation: RSV-HRP and RSV-Lucia appeared to grow more slowly than a wildtype virus. However, both were readily generated from cDNA, and stock titers of 5 million PFU/ml were easily produced without any optimization. It is important to realize that very few PFU are needed in an in vitro neutralization assay, so optimal virus growth rates and stock titers are not an impediment. It is, however, always recommendable to maintain virus stocks at low passage. This is because, unless certain fitness pressure is provided, many if not all RSV viruses modified to express a foreign gene will downregulate expression of the foreign gene after successive passages. In case of RSV-HRP, no extra precautions were taken, and different stocks readily generated levels of HRP more than adequate to obtain the results shown here.

RSV-HRP lacking G protein expression: As a variation of RSV-HRP, an RSV-HRP lacking the G protein ORF was developed. The resulting virus (RSV-HRP-AG) is equally effective as a method to determine the impact of antibodies or drugs on RSV replication. However, in contrast to RSV-HRP, which measures antibodies against both G and F protein, RSV-HRP-AG measures the impact of antibodies specific only for the F glycoprotein. This application is useful as the F protein is the most important, and sometimes the sole, target for anti-RSV measures.

In summary, a novel, HRP-based, rapid and inexpensive in vitro neutralization assay was developed that requires only a standard ELISA plate reader. The simplicity and low cost of the assay and required buffers, substrate, and equipment resources may allow for much wider application, including in low-resource settings and rural areas. As the HRP gene can be included in any virus species that can be engineered to contain foreign sequences, the HRP-based concept is applicable to many viruses that cause disease in animals and humans. In addition to in vitro neutralization, the core technology of determining virus replication levels via virally-expressed HRP, can also be used to screen for antiviral drugs. Incorporation of HRP into viral genomes can also be used for other assay types that measure viral activity, based on ELISA, TCID₅₀, plaque assay, and the like.

Thus, in accordance with the present disclosure, there have been provided compounds, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A composition, comprising:
a recombinant horseradish peroxidase (HRP) gene construct represented by one of SEQ ID NOS: 3-6.

2. The composition of claim 1, wherein the gene construct is represented by SEQ ID NO:6.

3. A recombinant vector comprising the composition of claim 1.

4. A recombinant viral genome comprising an open reading frame, the open reading frame comprising:
a nucleotide sequence encoding at least a portion of a horseradish peroxidase (HRP);
a nucleotide sequence flanking an amino terminal end of the HRP gene and encoding a heterologous signal peptide; and
a nucleotide sequence flanking a carboxyl terminal end of the HRP gene and encoding a heterologous transmembrane domain.

5. The recombinant viral genome of claim 4, wherein the open reading frame further comprises a nucleotide sequence encoding a myc epitope tag.

6. The recombinant viral genome of claim 4, wherein the nucleotide sequence encoding at least a portion of HRP has been codon optimized for mammalian expression.

7. The recombinant viral genome of claim 4, wherein at least one of:
the signal peptide comprises at least a portion of a signal peptide of nerve growth factor receptor (NGFR); and
the transmembrane domain comprises at least a portion of a transmembrane domain of NGFR.

8. The recombinant viral genome of claim 4, wherein at least a portion of the open reading frame is represented by one of SEQ ID NOS: 2-6.

9. The recombinant viral genome of claim 8, wherein the open reading frame is represented by SEQ ID NO:6.

10. A recombinant virus, comprising:
a modified horseradish peroxidase (HRP) gene construct incorporated in a genome of the virus, wherein the modified HRP gene construct comprises an open reading frame comprising:
a nucleotide sequence encoding at least a portion of a horseradish peroxidase (HRP);
a nucleotide sequence flanking an amino terminal end of the HRP gene and encoding a heterologous signal peptide; and
a nucleotide sequence flanking a carboxyl terminal end of the HRP gene and encoding a heterologous transmembrane domain.

11. The recombinant virus of claim 10, wherein the nucleotide sequence encoding at least a portion of HRP has been codon optimized for mammalian expression.

12. The recombinant virus of claim 10, wherein the open reading frame further comprises a nucleotide sequence encoding a myc epitope tag.

13. The recombinant virus of claim 10, wherein at least one of:
the signal peptide comprises at least a portion of a signal peptide of nerve growth factor receptor (NGFR); and
the transmembrane domain comprises at least a portion of a transmembrane domain of NGFR.

14. The recombinant virus of claim 10, wherein at least a portion of the open reading frame is represented by one of SEQ ID NOS: 2-6.

15. The recombinant virus of claim 14, wherein the open reading frame is represented by SEQ ID NO:6.

16. The recombinant virus of claim 10, wherein the virus is a respiratory syncytial virus (RSV).

17. The recombinant virus of claim 16, wherein the RSV lacks at least one of a gene encoding an SH protein and a gene encoding a G protein.

18. The recombinant virus of claim 10, wherein the HRP encoded by the open reading frame is a secreted HRP protein.

19. A method, comprising:
incorporating a modified horseradish peroxidase (HRP) gene construct into a viral genome to produce a modified viral genome.

20. The method of claim 19, wherein the modified HRP gene construct comprises an open reading frame comprising:
a nucleotide sequence encoding at least a portion of a horseradish peroxidase (HRP);
a nucleotide sequence encoding a heterologous signal peptide flanking an amino terminal end of the HRP gene; and
a nucleotide sequence encoding a heterologous transmembrane domain flanking a carboxyl terminal end of the HRP gene.

21. The method of claim 19, wherein the viral genome is an RSV genome.

22. The method of claim 21, wherein the RSV genome lacks genes encoding an SH protein and a G protein.

23. A kit, comprising:
at least one composition of claim 1 or 2;
at least one recombinant vector of claim 3;
at least one recombinant viral genome of any one of claims 4-9; and/or
at least one recombinant virus of any one of claims 10-18.

24. The kit of claim 23, further comprising a detectable substrate for HRP.

25. The kit of claim 23, wherein the detectable substrate for HRP is selected from the group consisting of o-phenylenediamine dihydrochloride (OPD); 3,3′,5,5′-tetramethylbenzidine (TMB); and 2,2′-azino-bis(3-ethylbenzothiazoline-6-sulfonic acid) (ABTS).

26. A method, comprising the steps of:
(i) contacting the recombinant virus of any one of claims 10-18 with at least one candidate agent and incubating under conditions that allow the at least one candidate agent to interact with the recombinant virus;
(ii) contacting the recombinant virus with at least one cell to form a mixture;
(iii) culturing the mixture under conditions that allow for infection of the at least one cell by the recombinant virus and expression by the at least one cell of the modified HRP from a genome of the recombinant virus;
(iv) contacting the at least one cell with a substrate for HRP;
(v) measuring an optical density (OD) to detect signal generated by the HRP substrate and calculating an HRP expression level based on the OD; and
(vi) determining that the at least one candidate agent is a potential antiviral agent if the HRP expression level observed in step (v) is lower than an HRP expression level obtained in the absence of the at least one candidate agent.

27. The method of claim 26, wherein the at least one candidate agent comprises an antibody.

28. The method of claim 26, wherein the HRP substrate is selected from the group consisting of o-phenylenediamine dihydrochloride (OPD); 3,3′,5,5′-tetramethylbenzidine (TMB); and 2,2′-azino-bis(3-ethylbenzothiazoline-6-sulfonic acid) (ABTS).

29. The method of claim 26, wherein the at least one cell is a HEp-2 cell.

30. A method, comprising the steps of:
(i) contacting the recombinant virus of any one of claims 10-18 with at least one candidate agent to form a suspension and incubating the suspension under conditions that allow the at least one candidate agent to interact with the recombinant virus;
(ii) contacting and culturing at least one cell attached to a surface with the suspension under conditions that allow for infection of the at least one cell by the recombinant virus;
(iii) substantially removing the suspension and culturing the at least one cell in the presence of growth medium under conditions that allow for expression of the modified HRP from a genome of the recombinant virus;
(iv) substantially removing a supernatant containing the growth medium and contacting the at least one cell with a substrate for HRP;
(v) measuring an optical density (OD) to detect signal generated by the HRP substrate and calculating an HRP expression level based on the OD; and
(vi) determining that the at least one candidate agent is a potential antiviral agent if the HRP expression level observed in step (v) is lower than an HRP expression level obtained in the absence of the at least one candidate agent.

31. The method of claim 30, wherein the at least one candidate agent comprises an antibody.

32. The method of claim 30, wherein the HRP substrate is selected from the group consisting of o-phenylenediamine dihydrochloride (OPD); 3,3′,5,5′-tetramethylbenzidine (TMB); and 2,2′-azino-bis(3-ethylbenzothiazoline-6-sulfonic acid) (ABTS).

33. The method of claim 30, wherein the at least one cell is a HEp-2 cell.

34. The method of claim 30, further comprising the step of adding a stop solution between steps (iv) and (v).

35. A method, comprising the steps of:
(i) contacting at least one cell with the recombinant virus of any one of claims 10-18 and at least one candidate agent to form a mixture;
(ii) culturing the mixture under conditions that allow for infection of the at least one cell by the recombinant virus and expression by the at least one cell of the modified HRP from a genome of the recombinant virus;
(iii) contacting the at least one cell with a substrate for HRP;
(iv) measuring an optical density (OD) to detect signal generated by the HRP substrate and calculating an HRP expression level based on the OD; and
(v) determining that the at least one candidate agent is a potential antiviral agent if the HRP expression level observed in step (v) is lower than an HRP expression level obtained in the absence of the at least one candidate agent.

36. The method of claim 35, wherein the at least one candidate agent is a small molecule drug.

37. The method of claim 35, wherein the at least one cell is contacted with the recombinant virus prior to the addition of the at least one candidate agent.

38. The method of claim 35, wherein the HRP substrate is selected from the group consisting of o-phenylenediamine dihydrochloride (OPD); 3,3′,5,5′-tetramethylbenzidine (TMB); and 2,2′-azino-bis(3-ethylbenzothiazoline-6-sulfonic acid) (ABTS).

39. The method of claim 35, wherein the at least one cell is a HEp-2 cell.

40. The method of claim 35, further comprising the step of adding a stop solution between steps (iii) and (iv).
